(19) United States
(12) Patent Application Publication
(10) Pub. No.: US 2020/0268990 A1
(43) Pub. Date: Aug. 27, 2020

(54) Inhaler Device

(71) Applicant: UNIVERSITY OF WALES TRINITY ST DAVID, West Glamorgan (GB)

(72) Inventor: Caerwyn Ash, Swansea (GB)

(73) Assignee: Caerwyn Ash, Swansea (GB)

(21) Appl. No.: 15/998,554

(22) PCT Filed: Feb. 15, 2017
(86) PCT No.: PCT/GB2017/050399

§ 371 Date: Aug. 15, 2018

(30) Foreign Application Priority Data
Feb. 15, 2016 (GB) 1602655.1

(51) Int. Cl. A61M 15/00

(52) U.S. Cl. CPC A61M 15/002; A61M 15/009; A61M 15/0021; A61M 2205/183; A61M 2205/215; A61M 2205/587; A61M 2205/3334; A61M 2205/52; A61M 2205/581; A61M 2205/583; A61M 2205/3584

(57) ABSTRACT

An inhaler device (100) for delivering an airborne medicament to a user is disclosed. The device comprises a housing (110) having a chamber (121) for receiving a source of medicament (140), an air flow passage (131) for communicating an air flow through the housing between a passage inlet (132) and a passage outlet (133), and a sensor (135) disposed in the air flow passage which is configured to sense a rate of flow of air along the passage. The device further comprises an orientation sensor (170) for determining an orientation and movement of the inhaler device during use, a processor (160) communicatively coupled with the sensor and orientation sensor for processing data received from the sensor and orientation sensor, and a storage device (162) for storing the data.

The present invention relates to an inhaler device.

Medical devices, specifically those for self-medication, such as inhalers, have remained largely unchanged over the last 50 years. The inhalers typically comprise an aerosol canister for delivering an air borne medicament to a user. The medicament is released by pressing the canister which opens a valve so that the medicament becomes released as a spray. The user is required to time the release of the medicament with an inhalation so that the spray becomes entrained with the inhalation.

However, it is found that most inhaler devices are used incorrectly. For example, inhaling at the required low flowrate to ensure optimal delivery of the medicament into the user is counterintuitive. Users typically inhale too rapidly which results in the majority of the medicament falling in their mouth and throat, and consequently offers no direct therapy to the lungs. It is also known that the deposition of the drug within the users' mouth and throat can result in unpleasant side effects, such as allergic rhinitis and candidiasis.

We have now devised an improved inhaler device.

According to the present invention, there is provided an inhaler device for delivering an airborne medicament to a user, the device comprising a housing having a chamber for receiving a source of medicament, an air flow passage for communicating an air flow through the housing between a passage inlet and a passage outlet, and a sensor disposed in the air flow passage which is configured to sense a rate of flow of air along the passage, the device further comprising an orientation sensor for determining an orientation and movement of the inhaler device during use, a processor which is communicatively coupled with the sensor and the orientation sensor for processing data received from the sensor and orientation sensor, and a storage device for storing the data.

In an embodiment, the sensor is disposed proximate the inlet to the passage. In an embodiment, the sensor may comprise at least two pressure sensors disposed along the passage for example, for sensing an air pressure at different locations along the passage.

In an alternative embodiment, the sensor may comprise a rotor which is arranged to rotate about a shaft in dependence of the air flow along the passage. The shaft may be arranged to extend along a longitudinal axis of the passage, such that the rotor extends in a plane substantially perpendicular to an axis of the passage. However, in an alternative embodiment, the shaft may be orientated at an acute angle with respect to an axis of the passage. In this respect, the rotor is arranged to rotate upon the shaft as a user draws air along the passage from the inlet to the outlet, when inhaling, and creates a uniform air flow along the passage. Similarly, the rotor is arranged to rotate as a user blows air along the passage from the outlet to the inlet. The rate of rotation is thus determined by the force exerted by the user when inhaling/blowing.

Preferably, the rotor is arranged to rotate in a clockwise direction and in an anti-clockwise direction about the shaft. The rotor is thus configured for bidirectional rotation upon the shaft.

In an embodiment, the inhaler device further comprises a transducer for generating an audible signal and/or a light emitting device for generating a visual signal, in dependence of the air flow rate along the passage.

In an embodiment, the device further comprises a receiver disposed in the passage for receiving a dispensing outlet of the source of medicament. The receiver comprises a nozzle for distributing medicament dispensed from the dispensing outlet into the passage. Preferably, the nozzle is arranged to direct the medicament dispensed by the source in a direction which is toward the outlet of the passage. The source may comprise a pressurised canister of a fluid medicament for example, and the dispensing outlet is arranged to open a valve associated with the canister upon moving the dispensing outlet relative to the canister, such as by moving the dispensing outlet into the canister. The nozzle is preferably arranged to generate a spray of medicament droplets into the passage.

In an embodiment, the chamber is separated from the air flow passage by a bulkhead which extends across the housing. The bulkhead comprises an aperture formed therein through which the source of medicament can extend, such that the source of medicament becomes supported by the bulkhead. Preferably, the source is sealed to the bulkhead via a seal which extends around a periphery of the aperture, and which acts to seal an interior of the chamber from the air flow passage.

In an embodiment, the outlet of the passage comprises a mouthpiece.

In an embodiment, the device further comprises an actuator for releasing the medicament from the source. The actuator may comprise a trigger pivotally coupled at proximal region thereof to the housing. A distal region of the trigger comprises an engagement portion which is inclined relative to the source, and which extends over the source. The engagement portion is arranged to contact the source, such that upon pulling the trigger, the engagement portion slides over the source and owing to the inclination of the engagement portion, causes the source to move relative to the dispensing outlet, to release medicament as a spray into the passage.

In an embodiment, the data received by the processor from the sensor may comprise data relating to the operating characteristics of the inhaler device, such as the time and date the air flow is sensed along the passage, the rate of air flow and a direction and duration of the air flow and the like. The device further comprises a connector, such as a USB connector, for communicatively coupling the device with a computer for example, for downloading the data to the computer. Preferably, the processor, storage device and connector are disposed within the chamber. In an embodiment, the connector may be slidably coupled to the housing so that the connector can be moved into and out from the chamber via a port disposed on the housing. Alternatively, or in addition thereto, the device may further comprise a wireless communications device for communicatively coupling the processor with a wireless communications network, such that the data can be downloaded to a remote computing device over a communications network.

In an embodiment, the inhaler device comprises a closure or cap or similar which is detachably coupleable with the housing so that the chamber can be opened and closed to permit the source of medicament to be replaced, for example.

Whilst the invention has been described above, it extends to any inventive combination of features set out above or in the following description. Although illustrative embodiments of the invention are described in detail herein with reference to the accompanying drawings, it is to be understood that the invention is not limited to these precise embodiments.

Furthermore, it is contemplated that a particular feature described either individually or as part of an embodiment can be combined with other individually described features, or parts of other embodiments, even if the other features and embodiments make no mention of the particular feature. Thus, the invention extends to such specific combinations not already described.

The invention may be performed in various ways, and, by way of example only, embodiments thereof will now be described, reference being made to the accompanying drawings in which:

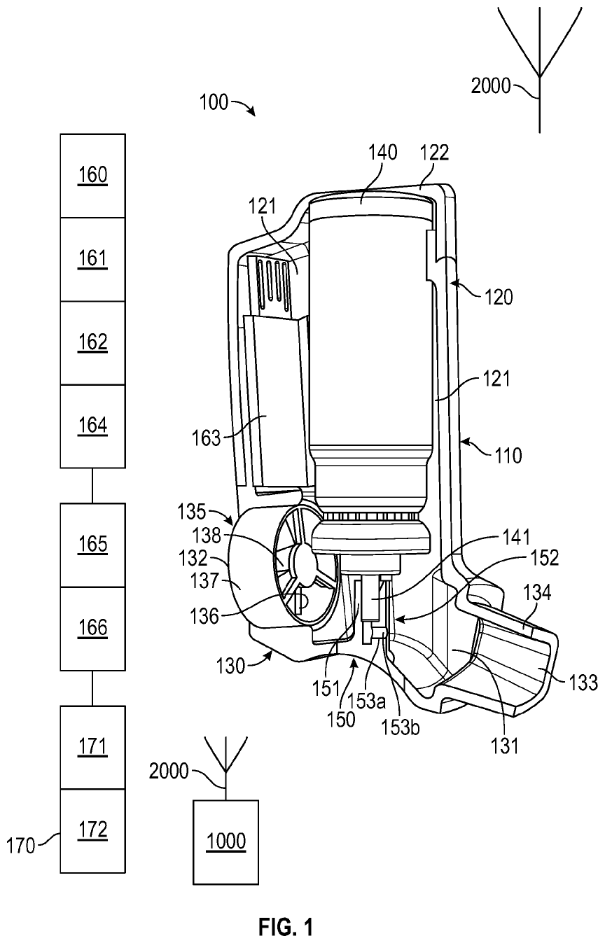

FIG. 1 is a sectional view of an inhaler device according to a first embodiment of the present invention; and,

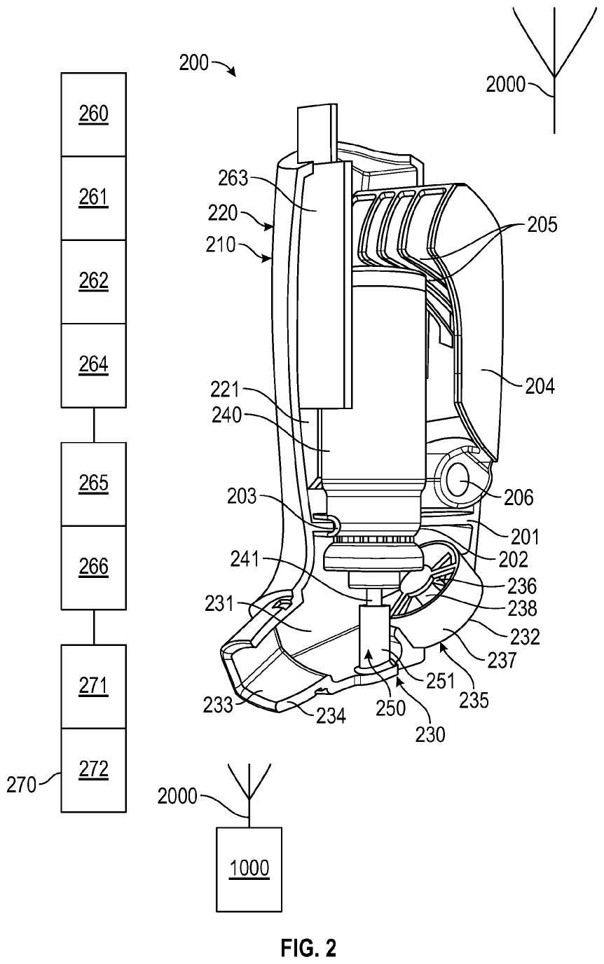

FIG. 2 is a sectional view of an inhaler device according to a second embodiment of the present invention.

Referring to FIG. 1 of the drawings, there is illustrated an inhaler device 100 according to a first embodiment of the present invention for delivering an airborne medicament to a user. The device comprises an elongate housing 110 which may be formed of a rigid plastics material for example, and comprises an upper and lower housing portion 120, 130 which may be formed integrally as a one piece unit, or may alternatively be formed separately and rigidly secured together.

The upper housing portion 120 defines an outer wall of a chamber 121 of the device 100 for receiving a source of medicament, which in the illustrated embodiment comprises a pressurised canister 140 of a fluid medicament. A cap or closure 122 may also be provided which is detachably coupleable at an upper region of the upper housing portion 120 so that the chamber 121 can be opened and closed to allow access within the chamber 121, such as when replacing a canister 140.

The lower housing portion 130 defines an outer wall of an air flow passage 131, which extends substantially transverse to a longitudinal axis of the upper housing portion 120, and in the illustrated embodiment, the chamber 121 and air flow passage 131 form a combined space within the housing 110. The air flow passage 131 comprises an inlet 132 thereto formed in the lower housing portion 130 and an outlet 133 disposed at an opposite end of the passage 131, which forms a mouthpiece 134 of the inhaler device 100 that extends away from the upper housing portion 120. The inlet and outlet 132, 133 allow a user to draw air along the passage 131 from the inlet 132 to the outlet 133, or alternatively blow air from the mouthpiece 134 along the passage, toward the inlet 132. The device 100 may further comprise a cover (not shown) for enclosing the mouthpiece 134 when the inhaler device 100 is not being used.

The air flow passage 131 further comprises a sensor 135, which in accordance with an embodiment, comprises at least two pressure sensors (not shown) disposed at separated locations along the air flow passage 131. The pressure sensors (not shown) are arranged to monitor a pressure of the air within the passage 131 and by comparing the sensed pressures of each sensor (not shown) it is possible to determine a speed of the air flow and a direction of the air flow within the passage 131.

In an alternative embodiment, the sensor 135 may comprise rotor assembly disposed proximate the inlet 132 which comprises a rotor 136 that is arranged to rotate in dependence of the air flow along the passage 131. The rotor 136 is housed within a casing 137 of the assembly and is permitted to rotate about a shaft 138 in both a clockwise and anti-clockwise direction in dependence of the direction of air flow along the passage 131. In an embodiment, the shaft 138 is orientated substantially collinearly with a longitudinal axis of the passage 131, such that the rotor 136 extends in a plane substantially transverse to the axis of the passage 131. However in alternative embodiments, the shaft 138 may instead be orientated at an acute angle with respect to the axis of the passage 131.

The air flow passage 131 further comprises a receiver 150 disposed therein at a position intermediate the inlet 132 and outlet 133 thereof, for receiving a dispensing outlet 141 of the canister 140. The receiver 150 comprises a substantially cylindrical housing 151 which extends from a lower portion of the passage 131 toward the chamber 121, substantially along an axis comprising a longitudinal axis of the chamber 121. The dispensing outlet 141 is arranged to locate within the receiver 150, through an open upper region thereof and form a friction fit therewith, when the canister 140 is located within the chamber 121. The receiver 150 further comprises a nozzle 152 formed therein, which comprises a narrow channel 153a that extends through a side wall 151a of the receiver housing 151 and which terminates at an opening 153b on an outer side of the side wall 151a housing 151, adjacent the mouthpiece 134. The channel 152 extends substantially parallel with an axis of the passage 131 and the opening 153 is arranged to direct the fluid medicament dispensed into the receiver 150 from the dispensing outlet 141, along the passage 131 toward the mouthpiece 134.

The inhaler device 100 further comprises a processor 160 (shown schematically in the drawings) disposed within the chamber 121, which is communicatively coupled with the sensor 135, such as the rotor assembly for receiving data relating to the operating characteristics of the rotor 136, such as activation time and date, speed and direction of air flow along the passage 131, duration of rotation and the like. In this respect, it is envisaged that the inhaler device 100 may further comprise a source of electrical power (not shown), such a battery source 161 (shown schematically in the drawings), for powering the processor 160. The data received by the processor 160 is stored in a storage device 162 (shown schematically in the drawings) which may also be disposed in the chamber 121. The data held within the storage device 162 may be downloaded to a computer 1000 via a connector 163, such as a USB connector, which may be slidably coupled to the housing 110, so that the connector 163 (shown schematically in FIG. 1) can slide into and out from the chamber 121 via a port (not shown) disposed within the upper housing portion 120 or closure 122, for example. Alternatively, or in addition thereto, the inhaler device 100 may further comprise a wireless communications device 164 so that the data can be downloaded from the storage device 162 to a remote computing device 1000 over a wireless communications network 2000. In a further embodiment, it is envisaged that the inhaler device 100 may further comprise an orientation sensor 170 comprising an accelerometer 171 and gyroscope 172 for determining an orientation and movement of the inhaler device 100 during use. The orientation sensor 170 is communicatively coupled with the processor 160 and is arranged to monitor a pitch for example, of the inhaler device 100 and any movement of the device 100 during use, so that the users style of operating the device 100 can be determined. It is envisaged that this may be useful for doctors and GP's for example, since the effectiveness of the medicament for the users medical condition can be assessed in comparison with the user's operation of the device 100.

Referring to FIG. 2 of the drawings, there is illustrated an inhaler device 200 according to a second embodiment of the present invention for delivering an airborne medicament to a user. The inhaler device 200 of the second embodiment comprises the same features as the inhaler device 100 of the first embodiment and so like features have been referenced using the same numerals but increased by 100.

The inhaler device 200 of the second invention however further comprises a bulkhead or partition 201 which extends across the housing 210 at the interface between the upper and lower housing portions 220, 230. The bulkhead 201 comprises an aperture 202 formed therein which comprises a seal 203 disposed around an inner periphery thereof. The aperture 202 is arranged to receive the canister 240 as the dispensing outlet 241 becomes received within the receiver 250. The seal 203 formed around the aperture 202 serves to form a secure fit with the canister 240, so that the canister 240 becomes supported by the bulkhead 201. With the canister 240 secured within the bulkhead 201, the seal 203 further forms a fluid seal with an outer surface of the canister 240, which effectively isolates the interior of the chamber 221 from the air flow passage 231, so that air cannot pass between the chamber 221 and the air flow passage 231.

The inhaler device 200 of the second embodiment further comprises an actuator 204, such as a trigger which is pivotally coupled at a proximal region thereof to the upper housing portion 220, via a pivot pin 206. The trigger 204 is disposed at a side of the inhaler device 200 disposed opposite the side from which the mouthpiece 234 extends, and is arranged to pivot in a plane comprising the mouthpiece 234. A distal region of the trigger 204 is arranged to extend over an upper region of the canister 240 and comprises an engagement portion 205 which is inclined relative to a longitudinal axis of the chamber 221, over the canister 240. The engagement portion 205 is arranged to contact an upper region of the canister 240 and as the trigger 204 is pulled, namely pivoted into the chamber 221, the engagement portion 205 is arranged to urge the canister 240 along the chamber 221 toward the air flow passage 231. The inclination of the engagement portion 205 creates a force component upon the canister 240 toward the receiver 250 as the trigger 204 is pulled, and thus causes the canister 240 to move relative to the dispensing outlet 241, which is held within the receiver 250. This action causes the fluid medicament to become dispensed into the receiver 250, and subsequently into the passage. In addition, the inclination of the engagement portion 205 imparts a lateral force upon the canister 240 as the trigger 204 is pulled, however this force component is resisted by the supporting bulkhead 201.

The inhaler device 100 of the first embodiment and the inhaler device 200 of the second embodiment can be used in two distinct modes of operation. For example, in a first mode when it is required to administer a medicament, a user removes the cover (not shown) enclosing the mouthpiece 134, 234 (and the closure 122 in the case of the inhaler device 100 of the first embodiment), places the mouthpiece 134, 234 in their mouth and inhales. Upon inhaling, air will become drawn into the passage 131, 231 whereupon the air flow will become sensed by the sensor 135, such as the rotor 136, 236. The air flow causes the rotor 136, 236 to rotate in a first direction, such as clockwise. The speed of rotation is dependent on the force exerted by the user when inhaling and as such, the preferred force can be indicated to the user as an audible signal generated by a transducer 165, 265 (shown schematically in the drawings), which may be disposed within the chamber 131, 231 and/or as a visual signal which may be generated by a light emitting device 166, 266 (shown schematically in the drawings), such as a light emitting diode

The transducer 165, 265 and/or light emitting device 166, 266 are communicatively coupled with the processor 160, 260, and the preferred inhalation force for suitably administering the medicament can be set as a preferred range of rotational speeds for the rotor. However, in the embodiment in which the sensor 135 comprises at least two pressure sensors (not shown) the air flow speed along the passage 131 will manifest as an air pressure variation along the passage. Accordingly, as the user inhales, the processor 160, 260 can monitor the rotational speed or the pressure variation, and when the rotational speed/pressure variation falls within the preferred range, then the processor 160, 260 is arranged to communicate a signal to the transducer 165, 265 and/or the light emitting device 166, 266 to cause an audible and/or visual signal to be generated, respectively. While the signal is being generated, the user directly presses the canister 140 into the chamber 121, in the case of the inhaler device 100 of the first embodiment, or pulls the trigger 204 of the inhaler device 200 of the second embodiment to cause the canister 140, 240 to move toward the lower housing portion 130, 230, and this action may be recorded by the processor 160, 260 to the storage device 162, 262. This causes the dispensing outlet 141, 241 to open a valve (not shown) associated with the canister 140, 240 so that the fluid medicament dispenses into the receiver 150, 250 and out into the passage 131, 231 via the nozzle 152, 252. The spray of medicament droplets within the passage 131, 231 subsequently become entrained within the air flow drawn along the passage 131, 231 during the inhalation and thus become suitably deposited at the desired location within the user. The inhaler device 100, 200 of the first and second embodiments thus record the users inspiratory flow profile; it can also deduce inspiratory power and energy, pressure drop profile, inhaled volume, peak inspiratory flowrate. Moreover, the processor 160 is configured to process the data received from the orientation sensor 170 to determine the orientation and movement of the device 100 during use, so that an assessment can be made whether the device 100 has been used correctly. All of these parameters can subsequently be analysed to assess lung function.

In a second mode of operation, the inhaler device 100, 200 of the first and second embodiments can be used to determine a peak flow measurement of the user, namely a user's lung capacity. This measurement is typically used by doctors and nurses when assessing a patient for asthma. In this mode of operation, the user removes the cover (not shown) enclosing the mouthpiece 134, 234 as before, places the mouthpiece 134, 234 in their mouth, but then instead of inhaling, the user blows into the mouthpiece 134, 234 as hard as they can. During this process, air will pass from the mouthpiece 134, 234, along the passage 131, 231 and out from the passage 131, 231 via the sensor 135, 235. In the embodiment where the sensor 135, 235 comprises a rotor assembly, then the air flow will cause the rotor 136, 236 to rotate in a second direction such as anti-clockwise and the speed with which the rotor rotates can be used to determine a peak flow measurement for the user. In this respect, the processor 160, 260 is arranged to monitor for the rotation of the rotor 136, 236, and record the rotation direction (to indicate whether a user is exhaling or inhaling) and the speed of rotation to the storage device 162, 262, in addition to the time and date, for example. Alternatively, in the situation where the sensor 135, 235 comprises at least two pressure sensors (not shown), then the speed, direction and duration of the air flow will manifest as a pressure variation along the passage 131 and this pressure variation can be used to extract the relevant operational characteristics in addition to the users peak flow measurement, for example.

The data acquired by the inhaler device 100, 200 in the first and second modes of operation is recorded to the storage device 162, 262 and can be downloaded to local computing device via the USB connector 163, 263, for example. However, it is envisaged that this download may also be achieved wirelessly, such as via a Bluetooth 6 connection. Alternatively, the data can be downloaded to a remote computing device or server over a wireless communications network 2000, via the communications device 164, 264. The inhaler devices 100, 200 of the first and second embodiments thus provide a complete record of the user's activity of the device 100, 200, including their operation of the device 100, 200 and can further determine when the canister 140, 240 may need replacing for example. If a user is required to administer a medicament at periodic times, then the inhaler device 100, 200 can also be arranged to provide audible and/or visual alerts to the user, via the transducer 165, 265 and/or light emitting device 166, 266.

From the foregoing therefore, it is evident that the inhaler device provides an improved device for administering an airborne medicament and can provide a complete record of a user's activity of the device.

What is claimed:

1. An inhaler device for delivering an airborne medicament to a user, the device comprising a housing having a chamber for receiving a source of medicament, an air flow passage for communicating an air flow through the housing between a passage inlet and a passage outlet, and a sensor configured to sense a rate of flow of air along the passage, the device further comprising an orientation sensor for determining an orientation and movement of the inhaler device during use, a processor communicatively coupled with the sensor and the orientation sensor for processing data received from the sensor and the orientation sensor, and a storage device for storing the data.

2. An inhaler device according to claim 1, wherein the sensor is disposed proximate the inlet to the passage.

3. An inhaler device according to claim 1, wherein the sensor comprises at least two pressure sensors disposed along the passage, for sensing an air pressure at different locations along the passage.

4. An inhaler device according to claim 1, wherein the sensor comprises a rotor which is arranged to rotate about a shaft in dependence of the air flow along the passage.

5. An inhaler device according to claim 4, wherein the shaft is arranged to extend along a longitudinal axis of the passage.

6. An inhaler device according to claim 4, wherein the shaft is orientated at an acute angle with respect to an axis of the passage.

7. An inhaler device according to claim 4, wherein the rotor is arranged to rotate in a clockwise direction and in an anti-clockwise direction about the shaft.

8. An inhaler device according to claim 1, further comprising a transducer for generating an audible signal and/or a light emitting device for generating a visual in dependence of the rate of air flow along the passage.

9. An inhaler device according to claim 1, further comprising a receiver disposed in the passage for receiving a dispensing outlet of the source of medicament.

10. An inhaler device according to claim 9, wherein the receiver comprises a nozzle for distributing medicament dispensed from the dispensing outlet into the passage.

11. An inhaler device according to claim 10, wherein the nozzle is arranged to direct the medicament dispensed by the source toward the outlet of the passage.

12. An inhaler device according to claim 1, wherein the chamber is separated from the air flow passage by a bulkhead which extends across the housing, the bulkhead comprising an aperture formed therein through which the source of medicament can extend, such that the source of medicament becomes supported by the bulkhead.

13. (canceled)

14. An inhaler device according to claim 12, wherein the source is sealed to the bulkhead via a seal which extends around a periphery of the aperture.

15. An inhaler device according to claim 1, wherein the outlet of the passage comprises a mouthpiece.

16. An inhaler device according to claim 1, further comprising an actuator for releasing the medicament from the source, the actuator comprising a trigger pivotally coupled at proximal region thereof to the housing.

17. (canceled)

18. An inhaler device according to claim 16, wherein a distal region of the trigger comprises an engagement portion which is inclined relative to the source, the engagement portion being arranged to contact the source, such that upon pulling the trigger, the engagement portion slides over the source and owing to the inclination of the surface portion, causes the source to move relative to the dispensing outlet.

19. (canceled)

20. An inhaler device according to claim 1, further comprising a connector, for communicatively coupling the inhaler device with a computer for downloading the data to the computer.

21. An inhaler device according to claim 20, wherein the processor, storage device and connector are disposed within the chamber.

22. (canceled)

23. An inhaler device according to claim 1, further comprising a wireless communications device for communicatively coupling the inhaler device with a wireless communications network.

24. An inhaler device according to claim 1, further comprising a closure which is detachably coupleable with the housing so that the chamber can be opened and closed.

25. (canceled)
